(19) United States
(12) Patent Application Publication
(10) Pub. No.: US 2004/0248928 A1
(43) Pub. Date: Dec. 9, 2004

(54) Pharmaceutical composition comprising an adenosine A1/A2 agonist and a sodium hydrogen exchanger inhibitor

(75) Inventors: James M. Downey, Mobile, AL (US); Zhelong Xu, Chapel Hill, AL (US); Umesh Shukla, Belle Mead, AL (US)

Correspondence Address: SYNNESTVEDT & LECHNER LLP, 2600 ARAMARK TOWER, 1101 MARKET STREET, PHILADELPHIA, PA, 19107-2950, US

(73) Assignee: Adventis Pharmaceuticals Inc., Bridgewater, NJ

(21) Appl. No.: 10/835,964

(22) Filed: Apr. 30, 2004

Related U.S. Application Data
(63) Continuation of application No. PCT/US02/35096, filed on Nov. 1, 2002.
(60) Provisional application No. 60/336,315, filed on Nov. 2, 2001.

(30) Foreign Application Priority Data
Feb. 15, 2002 (GB) 0203596.2

(51) Int. Cl. A61K 31/4745

(52) U.S. Cl. 514/303

(57) ABSTRACT

The invention is directed to pharmaceutical composition comprising a compound having adenosine A1/A2 agonistic activity, a sodium-hydrogen exchanger inhibitory compound and a pharmaceutically acceptable carrier. The invention is also directed to a method cardioprotection in a patient in need thereof comprising administering to said patient pharmaceutically effective amounts of a compound having adenosine A1/A2 agonistic activity and a sodium-hydrogen exchanger inhibitory compound. This invention is also directed to the use of pharmaceutically effective amounts of a compound having adenosine A1/A2 agonistic activity and a sodium-hydrogen exchanger inhibitory compound in the preparation of a medicament for providing cardioprotection to a patient in need thereof. This invention is also directed to a kit for providing cardioprotection in a patient in need thereof, said kit comprising a plurality of separate containers, wherein at least one of said containers contains a compound having adenosine A1/A2 agonistic activity and at least another of said containers contains a sodium-hydrogen exchanger inhibitory compound, and said containers optionally contain a pharmaceutical carrier.

This application is a continuation of International Application No. PCT/US02/35096, filed Nov. 1, 2002, which claims the benefit of U.S. Provisional Application No. 60/336,315, filed Nov. 2, 2001.

### FIELD OF THE INVENTION

This invention is directed to a pharmaceutical composition comprising a compound having adenosine A1/A2 agonistic activity and a sodium-hydrogen exchanger inhibitory compound which exhibits unexpectedly efficacious activity for cardioprotection in a patient in need thereof. The invention is also directed to a method of providing cardioprotection in a patient comprising administering pharmaceutically effective amounts of a compound having adenosine A1/A2 agonistic activity and a sodium-hydrogen exchanger inhibitory compound.

### SUMMARY OF THE INVENTION

This invention is directed to a pharmaceutical composition comprising a compound having adenosine A1/A2 agonistic activity, or a pharmaceutically acceptable salt thereof, and a sodium-hydrogen exchanger inhibitory compound, or a pharmaceutically acceptable salt thereof. The invention is also directed to a method of providing cardioprotection in a patient in need thereof comprising administering pharmaceutically effective amounts of a compound having adenosine A1/A2 agonistic activity and a sodium-hydrogen exchanger inhibitory compound.

### DETAILED DESCRIPTION OF THE INVENTION

As used above, and throughout the description of the invention, the following terms, unless otherwise indicated, shall be understood to have the following meanings:

“Patient” includes both human and other mammals.

“Effective amount” is meant to describe an amount of composition according to the present invention effective in producing the desired therapeutic effect.

“Cardioprotection” means protecting against or reducing damage to the myocardium, for example prior to, during or after an ischemic attack, during reperfusion, or prior to during or after cardiac surgery.

“Adenosine A1/A2 agonist” or “compound having adenosine A1/A2 agonistic activity” means a compound which is an agonist for both the A1 and A2 subtypes of adenosine receptors, for example, AMP 579.

“Sodium-hydrogen exchanger inhibitory compound” or “NHE inhibitor” means an inhibitor of sodium-hydrogen exchange system, a pH regulating cellular ion transport system. Examples of sodium-hydrogen exchanger inhibitory compounds include cariporide (Aventis), eniporide (Merck KGAA), zoniporide (Pfizer), BMS-284640 (Bristol-Myers Squibb), BIIB-513 (Boehringer Ingelheim), BIIB-722 CI (Boehringer Ingelheim), EMD-85131 (Merck KGAA), KB-R9032 (Kanebo), MS-31-038 (Mitsui), SL-59.1227 (Sanofi), SM20550 (Sumitomo), SMP-300 (Fukushima Medical College), T-559 (Takeda) and TY-12533 (To a Eiyo).

“AMP 579” is [1S-[1α,2β,3β,4α(S*)]]-4-[7-[[3-chloro-2-thienyl)methyl]propyl]amino]-3H-imidazo[4,5-b]pyridin-3-yl]-N-ethyl-2,3-dihydroxycyclopentanecarboxamide, or

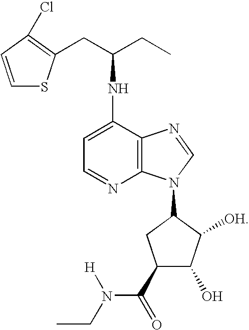

“Cariporide” is 4-isopropyl-3-methylsulfonylbenzoylguanidine methane sulfonate, or

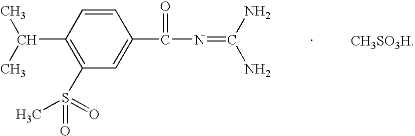

AMP 579, a new adenosine A₁/A₂ receptor agonist, has shown to be cardioprotective when administered at reperfusion. Pretreatment with the Na⁺/H⁺ exchanger inhibitor cariporide or ischemic preconditioning (PC) have also been demonstrated to limit infarct size. In the present study we investigated whether AMP 579's action at reperfusion can be added to the protective effect of either cariporide or PC. Open-chest rabbit hearts were subjected to 45 min regional ischemia followed by 3 h reperfusion. Infarct size in the control group was 55.8±3.9%. PC by 5 min ischemia+10 min reperfusion significantly reduced infarct size to 26.0±6.7%. AMP 579 was given as a bolus injection (30 ug/kg) followed by an infusion (3 ug/kg/min) for 70 min starting just before reperfusion. AMP 579 alone also significantly limited infarct size (32.1±1.8%). The combination of AMP 579 and PC showed a greater limitation of infarct size (5.5±2.7%) compared to either PC or AMP 579 alone. In a second series of studies, the hearts were subjected to 60 min regional ischemia followed by 3 h reperfusion. Infarct size in the control group was 66.0±4.9%. A bolus injection of cariporide (0.5 mg/kg) 5 min prior to the onset of ischemia significantly reduced infarct size to 41.5±7.7%. When cariporide was combined with AMP 579, infarction was further limited to a markedly small size (14.2±4.5%). Although there was a trend toward protection with AMP 579 alone (45.3±5.4%) it was not significant suggesting that there is a limit to the ischemic insult against which AMP579 can protect. The combination of AMP 579 with a bolus injection of cariporide just before reperfusion, however, did significantly limit infarct size (31.3±7.0%). These results indicate that AMP 579's action at reperfusion can be synergistic to the protective effect conferred either by cariporide or PC.

The novel adenosine A₁/A₂ receptor agonist AMP 579 can protect the heart from ischemia/reperfusion injury in a variety of animal species when administered just before reperfusion. (Smits G J, McVey M, Cox B F, Perrone M H, Clark K L: Cardioprotective effects of the novel adenosine A₁/A₂ receptor agonist AMP 579 in a porcine model of myocardial infarction. J Pharmacol Exp Ther 1998;286:611-618 (hereinafter, “Smits”); McVey M J, Smits G J, Cox B F, Kitzen J M, Clark K L, Perrone M H: Cardiovascular pharmacology of the adenosine A₁/A₂-receptor agonist AMP 579: coronary hemodynamic and cardioprotective effects in the canine myocardium. J Cardiovasc Pharmacol 1999;33:703-710 (hereinafter, “McVey”); Budde J M, Velez D A, Zhao Z-Q, Clark K L, Morris C D, Muraki S, Guyton R A, Vinten-Johansen J: Comparative study of AMP579 and adenosine in inhibition of neutrophil-mediated vascular and myocardial injury during 24 h of reperfusion. Cardiovasc Res 2000;47:294-305 (hereinafter, “Budd”); and Xu Z, Yang X-M, Cohen M V, Neumann T, Heusch G, Downey J M: Limitation of infarct size in rabbit hearts by the novel adenosine receptor agonist AMP 579 administered at reperfusion. J Mol Cell Cardiol 2000;32:2339-2347(hereinafter, “Xu”)). AMP 579's protection at reperfusion may be attributable to reduction in myocardial contracture (Xu Z, Downey J M, Cohen M V: AMP 579 reduces contracture and limits infarction in rabbit heart by activating adenosine A₂ receptors. J Cardiovasc Pharmacol 2001, in press (hereinafter, “Xu II”)) and perhaps attenuation of free radical generation upon reperfusion (Xu Z, Cohen M V, Downey J M, Vanden Hoek T L, Yao Z: Attenuation of oxidant stress during reoxygenation by AMP 579 in cardiomyocytes. Am J Physiol 2001; submitted (hereinafter Xu III)). Because AMP 579 can be given just before reperfusion to protect the heart, it is reasonable to speculate that the mechanism AMP 579's protection is fundamentally different from interventions that must be given as a pretreatment such as cariporide or ischemic preconditioning.

Cariporide, a selective inhibitor of the subtype-i sodium-hydrogen exchanger (NHE-1), has also been demonstrated to protect heart from ischemia/reperfusion injury in a variety of experimental models (Miura T, Ogawa T, Suzuki K, Goto M, Shimamoto K: Infarct size limitation by a new Na⁺-H⁺ exchange inhibitor, Hoe 642: difference from preconditioning in the role of protein kinase C. J Am Col Cardiol 1997;29:693-701(hereinafter, “Miura”); Scholz W, Albus U, Counillon L, Gögelein H, Lang H-J, Linz W, Weichert A, Schölkens B A: Protective effects of HOE642, a selective sodium-hydrogen exchange subtype 1 inhibitor, on cardiac ischaemia and reperfusion. Cardiovasc Res 1995;29:260-268 (hereinafter, “Scholz”); Klein H H, Bohle R M, Pich S, Lindert-Heimberg S, Wollenweber J, Schade-Brittinger C, Nebendahl K: Time-dependent protection by Na⁺/H⁺ exchange inhibition in a regionally ischemic, reperfused porcine heart preparation with low residual blood flow. J Mol Cell Cardiol 1998;30:795-801 (hereinafter, “Klein”); Ito Y, Imai S, Ui G, Nakano M, Imai K, Kamiyama H, Naganuma F, Matsui K, Ohashi N, Nagai R: A Na+-H+ exchange inhibitor (SM-20550) protects from microvascular deterioration and myocardial injury after reperfusion. Eur J Pharm 1999;374:355 -366 (hereinafter, “Ito”)). Cariporide protects hearts by inhibiting an increase in intracellular sodium and subsequent intracellular calcium overload in the setting of myocardial ischemia/reperfusion, although it is still unclear whether cariporide confers its protection during ischemia or upon reperfusion the majority of studies indicate that it is most protective when given as a pretreatment (Gumina R J, Buerger E, Eickmeier C, Moore J, Daemmgen J, Gross G J: Inhibition of the Na+/H+ exchanger confers greater cardioprotection against 90 minutes of myocardial ischemia than ischemic preconditioning in dogs. Circulation. 1999;100:2519-2526 (hereinafter, “Gumino”); Klein H H, Pich S, Bohle R M, Wollenweber J, Nebendahl K: Myocardial protection by Na⁺-H⁺ exchange inhibition in ischemic, reperfused porcine hearts. Circulation. 1995;92:912-917 (hereinafter, “Klein II”); Rohmann S, Weygandt H, Minck K-O: Preischaemic as well as postischaemic application of a Na+/H+ exchange inhibitor reduces infarct size in pigs. Cardiovasc Res 1995;30:945-951 (hereinafter, “Rohmann”)).

Ischemic preconditioning (PC) is a phenomenon whereby exposure of the myocardium to a brief episode of ischemia and reperfusion markedly reduces tissue necrosis induced by a subsequent prolonged ischemia (Murry C E, Jennings R B, Reimer K A: Preconditioning with ischemia: a delay of lethal cell injury in ischemic myocardium. Circulation. 1986;74:1124-1136). PC is triggered by substances released during short periods of ischemia, including adenosine (Liu G S, Thornton J, Van Winkle D M, Stanley A W H, Olsson R A, Downey J M: Protection against infarction afforded by preconditioning is mediated by A₁ adenosine receptors in rabbit heart. Circulation. 1991;84:350-356(hereinafter, “Liu”)), bradykinin (Goto M, Liu Y, Yang X-M, Ardell J L, Cohen M V, Downey J M: Role of bradykinin in protection of ischemic preconditioning in rabbit hearts. Circ Res 1995;77:611-621(hereinafter, “Goto”)) and opioids (Schultz J E J, Rose E, Yao Z, Gross G J: Evidence for involvement of opioid receptors in ischemic preconditioning in rat hearts. Am J Physiol 1995;268:H2157-H2161), which are believed to subsequently activate protein kinase C (PKC) during a sustained ischemia (Ytrehus K, Liu Y, Downey J M: Preconditioning protects ischemic rabbit heart by protein kinase C activation. Am. J. Physiol. 1994;266:H1145-H1152 (hereinafter, “Ytrehus”); Weinbrenner C, Liu G-S, Cohen M V, Downey J M: Phosphorylation of tyrosine 182 of p38 mitogen-activated protein kinase correlates with the protection of preconditioning in the rabbit heart. J Mol Cell Cardiol 1997;29:2383-2391). The events happening downstream of PKC have not yet been well clarified. By definition PC must be given as a pretreatment in order to protect.

The mechanisms of protection likely differ among the above three interventions (AMP 579, cariporide and PC). If so then it should be possible that the drugs can be combined resulting in synergistic effects. Thus the present study aimed to investigate whether AMP 579's action at reperfusion can be synergistic to the protection induced either by cariporide or PC.

Some of the compounds comprising the compositions of the present invention having adenosine A1/A2 agonistic activity, or sodium-hydrogen exchanger inhibitory activity are basic, and such compounds are useful in the form of the free base or in the form of a pharmaceutically acceptable acid addition salt thereof.

The acids which can be used to prepare the acid addition salts include preferably those which produce, when combined with the free base, pharmaceutically acceptable salts, that is, salts whose anions are non-toxic to the patient in pharmaceutical doses of the salts, so that the beneficial effects inherent in the free base are not vitiated by side effects ascribable to the anions. Pharmaceutically acceptable salts within the scope of the invention include those derived from mineral acids and organic acids, and include hydrohalides, e.g. hydrochlorides and hydrobromides, sulfates, phosphates, nitrates, sulfamates, acetates, citrates, lactates, tartrates, malonates, oxalates, salicylates, propionates, succinates, fumarates, maleates, methylene-bis-b-hydroxynaphthoates, gentisates, isethionates, di-p-toluoyltartrates, methane-sulfonates, ethanesulfonates, benzenesulfonates, p-toluenesulfonates, cyclohexylsulfamates and quinates.

### Materials and Methods

This study was performed in accordance with The Guide for the Care and Use of Laboratory Animals (National Academy Press, Washington, D.C., 1996).

Surgical Preparation

New Zealand White rabbits of either sex weighing 2.0-2.5 kg were anesthetized with pentobarbital (30 mg/kg iv), intubated through a tracheotomy, and ventilated with 100% oxygen via a positive pressure respirator (MD industries, Mobile, Ala.). The ventilation rate and tidal volume were adjusted to maintain arterial blood gases in the physiological range. Body temperature was maintained at 38-39° C. A catheter was inserted into the left carotid artery for monitoring blood pressure. Another catheter was inserted into the right jugular vein for drug infusion. A left thoracotomy was performed in the fourth intercostal space, and the pericardium was opened to expose the heart. A 2-0 silk suture on a curved taper needle was passed through the myocardium around a prominent branch of the left coronary artery. The ends of the suture were passed through a small piece of soft vinyl tubing to form a snare. Ischemia was induced by pulling the snare and then fixing it by clamping the tube with a small hemostat. Ischemia was confirmed by appearance of cyanosis. Reperfusion was achieved by releasing the snare and was confirmed by visible hyperemia on the ventricular surface.

After 3 h of reperfusion, the rabbit was given an overdose of pentobarbital and the heart was quickly removed from the chest, mounted on a Langendorff apparatus, and perfused with saline to wash out blood. Then the coronary artery was reoccluded, and 1 ml of 0.25% fluorescent polymer microspheres (2-9 μm diameter, Duke Scientific Corp, Palo Alto, Calif.) were infused into the perfusate to demarcate the risk zone as the area of tissue without fluorescence. The heart was weighed, frozen, and cut into 2.5-mm-thick slices. The slices were incubated in 1% triphenyltetrazolium chloride (TTC) in sodium phosphate buffer at 37° C. for 20 min. The slices were immersed in 10% formalin to enhance the contrast between stained (viable) and unstained (necrotic) tissue and then squeezed between glass plates spaced exactly 2 mm apart. The myocardium at risk was identified by illuminating the slices with ultraviolet light. The infarcted and risk zone areas were traced on a clear acetate sheet and quantified with planimetry by an investigator blinded to the treatment. The areas were converted into volumes by multiplying the areas by slice thickness. Infarct size is expressed as a percentage of the risk zone.

Experimental Protocols

45 Minute Ischemia Model

Seven groups of rabbits were subjected to 45 min of regional ischemia followed by 3 h of reperfusion (FIG. 1). The PC was induced by a 5 min of regional ischemia followed by a 10 min reperfusion prior to the sustained ischemia. All groups receiving AMP 579 received a bolus injection of 30 μg/kg iv followed by an infusion of 3 μg/kg/min for 70 min. In PC+AMP (L) group, the hearts experienced PC and were treated with AMP 579 at reperfusion for 70 min. AMP 579 alone was given at reperfusion for 70 min in AMP (L) group. The hearts in AMP (E&L) group were treated with AMP 579 starting 5 min before ischemia for 120 min. An intravenous bolus injection of cariporide was given either 5 min prior to ischemia (Cariporide (E)) or 5 min before reperfusion (Cariporide (L)).

60 Minute Ischemia Model

Pretreatment with cariporide was so potent that any additional protection would probably be undetectable with a 45-minute ischemic insult. We therefore chose a 60 min period of index ischemia for these studies. As shown in FIG. 2, five groups of rabbits were subjected to 60 min of regional ischemia followed by 3 h of reperfusion. The control group received no drug treatment. All groups receiving AMP 579 received a bolus injection of 30 μg/kg iv followed by an infusion of 3 μg/kg/min for 70 min. In cariporide (E) group, the heart received a bolus injection of 0.5 mg/kg cariporide 5 min prior to ischemia. In cariporide (E)+AMP (L) group, in addition to the pretreatment with cariporide, the heart received AMP 579 at onset of reperfusion. The heart in cariporide (L)+AMP (L) group was treated with both cariporide (0.5 mg bolus) and AMP 597 at onset of reperfusion. In AMP (L) group, AMP 579 alone was administered at the onset of reperfusion.

Chemicals

AMP 579 and cariporide were obtained from Aventis Pharma and dissolved in small volumes of dimethylsulfoxide (DMSO) which had no independent effect on infarction.

Statistics

All data are expressed as means±S.E.M. One-way ANOVA combined with Scheffé's post hoc test was used to test for differences in baseline hemodynamics and infarct size among groups. ANOVA with replication was used to test for changes in hemodynamics during an experiment within each group. A p value of less than 0.05 was considered to be significant.

### Results

45 Minute Ischemia Model

In this model, we tested to see if the protective effect of AMP 579 can be added to that of PC. FIG. 3 reveals that one cycle of PC significantly limited infarct size from 55.8±3.9% of the risk zone in control animals to 26.0±6.7% of risk zone. Treatment with AMP 579 starting at reperfusion alone also significantly reduced infarct size to 32.1±1.8% of the risk zone. The combined use of PC with AMP 579 at reperfusion further reduced infarct size to 5.5±2.7% of the risk zone that was significantly smaller than that seen in either PC or AMP 579 alone. Thus an additive effect of AMP 579 and PC was seen. We also assessed the effect of cariporide on infarct size in the 45-minute ischemia model. Pretreatment with cariporide greatly reduced infarct size to 8.5±3.7% of the risk zone which was again smaller than that seen in the control hearts. However, when cariporide was administered just prior to reperfusion it failed to protect the hearts (53.4±3.5% infarction of the risk zone). Baseline heart rate and mean arterial pressure were not different among the five groups (Table 1). There were no significant differences in body weight, heart weight and risk zone size among the groups (Table 1).

60 Minute Ischemia Model

Because cariporide alone was so protective in the 45 min model we chose a 60 min index ischemia for the cariporide plus AMP579 protocols. Baseline heart rate and mean arterial pressure were not different among the five groups (Table 3). There were no significant differences in body weight, heart weight and risk zone size among the groups (Table 4). Infarct size in the control hearts was 66.0±4.9% of the risk zone (FIG. 4). Early treatment with cariporide significantly reduced infarct size to 41.5±7.7% of the risk zone. When the early administration of cariporide was combined with the with AMP 579 at reperfusion, the infarct size was further reduced to 14.2±4.5% of risk zone, indicating an synergistic effect of cariporide and AMP 579 on myocardial infarction. Administration of AMP 579 alone at reperfusion showed a trend for a smaller infarct size (45.3±5.4% of the risk zone) but statistical analysis revealed that the difference was not significant when compared to the control. Interestingly when both AMP 579 and cariporide were combined just prior to reperfusion the combination did significantly limit infarct size to 31.3±7.0% of the risk zone again suggesting some synergistic effect of cariporide and AMP 579 even when both were given at reperfusion.

### Discussion

The major finding of this study is that the protection of AMP 579 at reperfusion can be added to the protective effect of cariporide given before ischemia resulting in a profound level of protection. Another synergistic effect was seen when AMP 579 was combined with ischemic preconditioning. These findings suggest that the mechanism for the AMP 579's action is different from those for either cariporide or PC. Moreover, the combination of these drugs appear to provide a remarkable degree of protection against myocardial infarction in the clinical setting and may be particularly useful in cardiac surgery.

AMP 579 has been demonstrated to protect the heart against ischemia and reperfusion injury when administered at reperfusion (Smits; McVey; Budde; Xu), implying that AMP 579 can prevent reperfusion injury. In these studies, the hearts were subjected to 30 min ischemia and AMP 579 was administered either at 10 min before or onset of 3 h reperfusion. In the present study AMP on its own was protective in the 45 min model but protection could not be demonstrated in the 60 min model suggesting that there is an upper limit to the severity of the ischemic insult against which AMP can protect. While AMP 579's ability to protect at reperfusion is clearly less potent than that from cariporide pretreatment, it is remarkable that the combined effect was very dramatic indicating a synergistic effect.

In 45 min ischemia model of the present study, the late administration of cariporide alone (at reperfusion) was not cardioprotective at all (FIG. 4). That confirms the observation of others that also failed to protection when cariporide was introduced at reperfusion (Klein II; Klein H H, Pich S, Bohle R M, Lindert-Heimberg S, Nebendahl K: Na(+)/H(+) exchange inhibitor cariporide attenuates cell injury predominantly during ischemia and not at onset of reperfusion in porcine hearts with low residual blood flow. Circulation. 2000;102:1977-1982(hereinafter, “Klein III”). Interestingly, when the combination of cariporide and AMP 579 were administered at reperfusion, a significant decrease in infarct size was observed as compared to the control. While the protection was much less than that observed when cariporide was given as a pretreatment it does suggest some small effect at reperfusion. It is unclear, however, whether the protection is obtained through a “facilitation” mechanism or some additive effect.

Although the exact reason for the synergistic actions of AMP 579 and cariporide is unknown, we would speculate that the quite different mechanisms by which the two drugs act may result in the synergistic effect. AMP 579's protection at reperfusion seems to be mediated via stimulation of adenosine A₂ receptor (McVey; Xu II; Nakamura M, Zhao Z-Q, Clark K L, Velez D V, Guyton R A, Vinten-Johansen J: A novel adenosine analog, AMP579, inhibits neutrophil activation, adherence and neutrophil-mediated injury to coronary vascular endothelium. Eur J Pharmacol 2000;397:197-205 (hereinafter, “Nakamura”)). Our recent data also indicate that AMP 579 protects the heart from reperfusion injury through attenuation of myocardial contracture (Xu II) and it suppresses the burst of free radicals seen at reperfusion (Xu III). Nakamura et al. (Nakamura) proposed that suppression of neutrophil activation is involved in AMP 579's action. However, we found that AMP 579 was just as protective in buffer perfuse rabbit hearts which are neutrophil-free (Xu). Thus the exact mechanism of AMP 579's protection remains enigmatic. Cariporide is a selective NHE-1 inhibitor (Scholz). During ischemia accumulation of protons activates Na⁺/H⁺ exchanger and subsequently the Na⁺/H⁺ exchanger exchanges those for Na⁺. Accumulation of Na⁺ during ischemia interferes with volume control and at reperfusion Na⁺ can exchange with Ca⁺⁺ leading to cytosolic calcium overload. At reperfusion when pH is normalized the NHE-1 should be particularly active. Although NHE-1 inhibition has been widely recognized to be cardioprotective (Gumina; Klein III; Rupprecht H J, Dahl J V, Terres W, Seyfarth K M, Richardt G, Schultheib H P, Buerke M, Sheehan F H, Drexler H: Cardioprotective effects of the Na(+)/H(+) exchange inhibitor cariporide in patients with acute anterior myocardial infarction undergoing direct PTCA. Circulation. 2000; 101:2902-2908; Stromer H, de Groot M, Horn M, Faul C, Leupold A, Morgan J P, Scholz W, Neubauer S: Na⁺/H⁺ exchange inhibition with HOE642 improves postischemic recovery due to attenuation of Ca²⁺ overload and prolonged acidosis on reperfusion. Circulation 2000;101:2749-2755), it is still unclear whether the protection is exerted during ischemia (Miura; Klein; Klein II) or at reperfusion (Rohmann; Linz W, Albus U, Crause P, Jung W, Weichert A, Scholkens B A, Scholz W: Dose-dependent reduction of myocardial infarct mass in rabbits by the NHE-1 inhibitor cariporide (HOE 642). Clin Exp Hypertens 1998;20:733-749). In a recent report, Klein et al. has addressed that myocardial protection by cariporide is predominantly achieved by NHE-1 inhibiting during ischemia and not during reperfusion (Klein III). In agreement with this report, we also found that cariporide treatment at reperfusion could not protect the heart from ischemia/reperfusion injury in 45 min model. Thus it is reasonable to assume that different mechanisms are involved in the action of AMP 579 and cariporide. The different mechanisms of the two drugs are likely the basis of the synergistic effect.

In 45 min ischemia model of the present study, the protective effect of AMP 579 was also added to that of PC. PC is triggered by substances released during short periods of ischemia, including adenosine, bradykinin and opioids (Liu; Goto), which are believed to subsequently activate protein kinase C (PKC) during a sustained ischemia (Ytrehus). Using some specific antagonists, it has been well established that A₁ and A₃ but not A₂ adenosine receptors could initiate the protection of ischemic preconditioning (Thornton J D, Liu G S, Olsson R A, Downey J M: Intravenous pretreatment with A₁-selective adenosine analogues protects the heart against infarction. Circulation. 1992;85:659-665; Liu G S, Richards S C, Olsson R A, Mullane K, Walsh R S, Downey J M: Evidence that the adenosine A₃ receptor may mediate the protection afforded by preconditioning in the isolated rabbit heart. Cardiovasc Res 1994;28:1057-1061). When administered at reperfusion, AMP 579 protects the heart against ischemia/reperfusion injury through activation of A₂ but not A₁ receptors (Xu; Nakamura), indicating a difference in the mechanism between AMP 579 and PC. Thus, it is not surprising that AMP 579 at reperfusion can be additive with the protection of PC.

When given before ischemia, we speculated that AMP 579 might effect its protection by stimulating adenosine A₁ receptors and invoke the mechanism of PC (McVey). If it is the case, administration of AMP 579 starting prior to ischemia lasting until after 70 min of reperfusion should be expected to produce a level of protection comparable with that of PC plus AMP 579. However, we failed to observe this “expected” effect in our 45 min ischemia model (FIG. 4). It is possible that AMP579 was not given in high enough concentration to get adequate A₁ receptor stimulation to precondition the heart.

In summary, we have demonstrated that the application of the novel adenosine A₁/A₂ receptor agonist AMP 579 in combination with either cariporide or ischemia preconditioning greatly attenuated myocardial infarct size in open-chest rabbit hearts. The difference in the mechanisms among the three interventions may contribute to the additive effects. Furthermore, the present findings may provide a highly potent means of protecting the heart during cardiac surgery where pretreatment is an option.

### FIGURE LEGENDS

FIG. 1 Experimental protocols for 45 min ischemia model.

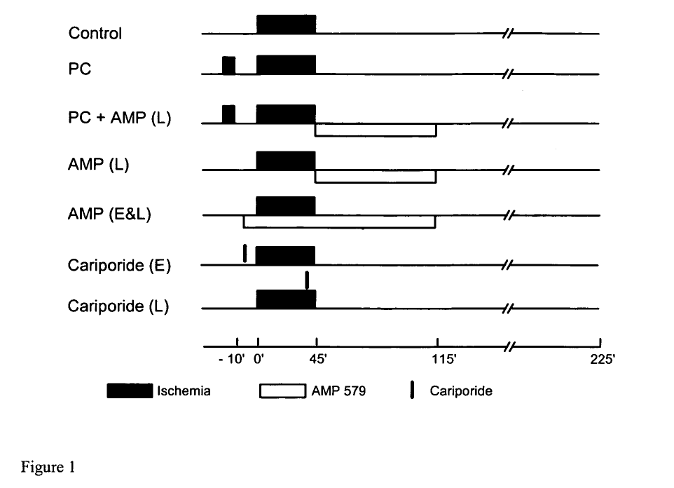

FIG. 2 Experimental protocols for 60 min ischemia model.

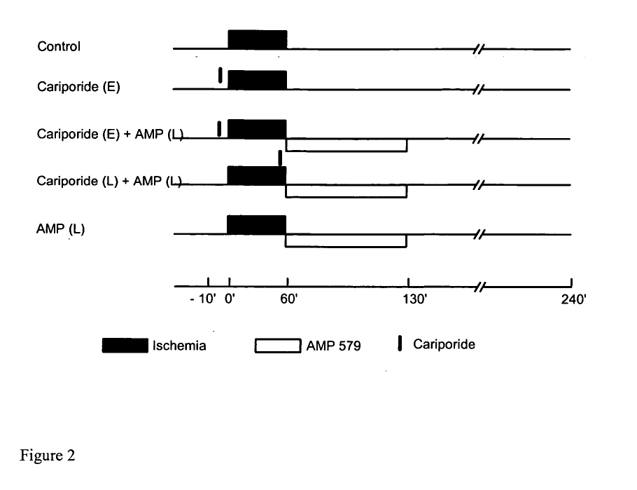

FIG. 3 Effects of PC and AMP 579 on myocardial infarct size expressed as a percentage of the risk zone. Infarct size was quantitated with triphenyltetrazolium (TTC) staining. Open circles represent individual experiments while closed circles depict group means with S.E.M. * p<0.05 vs. control; # p<0.05 vs. PC and AMP (L).

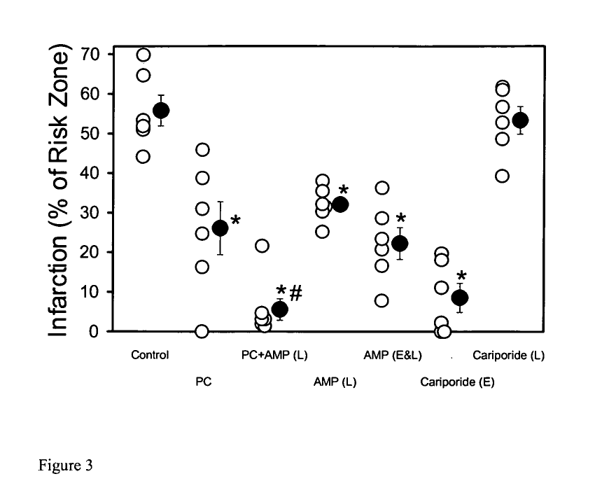

FIG. 4 Effects of cariporide and AMP 579 on myocardial infarct size expressed as a percentage of the risk zone in 60 min ischemia model. Infarct size was quantitated with triphenyltetrazolium (TTC) staining. Open circles represent individual experiments while closed circles depict group means with S.E.M. Abbreviations: see Table 1. * p<0.05 vs. control; # p<0.05 vs. cariporide (E).

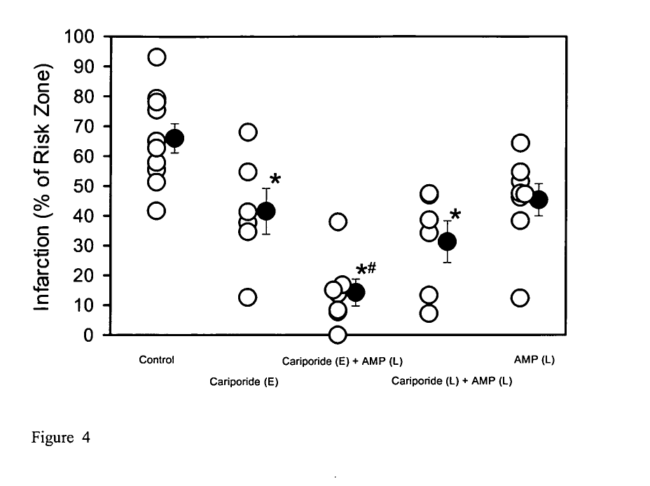

TABLE 1
Hemodynamic data for 45 min ischemia model

| | | | | |
|---|---|---|---|---|
| Baseline | Ischemia | Rep 30′ | Rep 90′ | Rep 180′ |

HR
(beats/min)

| | | | | | |
|---|---|---|---|---|---|
| Control | 260 ± 10 | 265 ± 13 | 253 ± 10 | 247 ± 14 | 248 ± 15 |
| PC | 278 ± 10 | 280 ± 10 | 273 ± 9 | 275 ± 10 | 270 ± 11 |
| PC + | 276 ± 9 | 269 ± 8 | 240 ± 12 | 233 ± 5 | 246 ± 10 |
| AMP (L) | | | | | |
| AMP (L) | 267 ± 6 | 265 ± 6 | 230 ± 7 | 247 ± 13 | 247 ± 8 |
| AMP (E&L) | 288 ± 8 | 247 ± 11 | 238 ± 12 | 233 ± 10 | 257 ± 11 |
| Cariporide | 290 ± 9 | 275 ± 9 | 273 ± 8 | 267 ± 8 | 260 ± 7 |
| (E) | | | | | |
| Cariporide | 277 ± 10 | 263 ± 11 | 258 ± 9 | 257 ± 8 | 248 ± 6 |
| (L) | | | | | |
| MAP | | | | | |
| (mmHg) | | | | | |
| Control | 96.3 ± 4.5 | 81.4 ± 5.0 | 79.1 ± 4.9 | 78.6 ± 4.8 | 78.0 ± 3.1 |
| PC | 100 ± 3.9 | 88.1 ± 2.5 | 88.1 ± 2.7 | 86.9 ± 3.4 | 80.0 ± 2.5 |
| PC + | 101 ± 3.9 | 87.9 ± 3.0 | 70.5 ± 7.7 | 73.3 ± 7.2 | 82.2 ± 7.9 |
| AMP (L) | | | | | |
| AMP (L) | 92.8 ± 5.6 | 72.2 ± 2.6 | 58.9 ± 4.6 | 67.8 ± 6.2 | 67.3 ± 4.9 |
| AMP (E&L) | 93.9 ± 4.6 | 65.8 ± 4.9 | 62.8 ± 4.3 | 67.2 ± 3.8 | 77.2 ± 4.9 |
| Cariporide | 95.6 ± 3.7 | 84.2 ± 5.7 | 83.9 ± 4.3 | 83.4 ± 4.6 | 78.2 ± 5.3 |
| (E) | | | | | |
| Cariporide | 93.9 ± 2.7 | 75.5 ± 4.1 | 73.6 ± 3.2 | 77.5 ± 2.3 | 71.9 ± 4.5 |
| (L) | | | | | |

Mean ± S.E.M.
Abbreviations:
PC = ischemic preconditioning;
PC + AMP (L) = ischemic preconditioning + administration of AMP 579 starting at reperfusion for 70 min;
AMP (L) = administration of AMP 579 starting at reperfusion for 70 min;
AMP (E&L) = administration of AMP 579 starting 5 min prior to ischemia lasting for 120 min;
Cariporide (E) = a bolus injection of cariporide 5 min prior to ischemia;
Cariporide (L) = a bolus injection of cariporide 5 min prior to reperfusion;
HR = heart rate;
MAP = mean arterial pressure.

TABLE 2
Infarct size data for 45 min ischemia model

| | | | | Risk zone | Infarct size |
|---|---|---|---|---|---|
| | n | Body weight (kg) | Heart weight (g) | (cm³) | (cm³) |
| Control | 6 | 2.3 ± 0.1 | 7.2 ± 0.3 | 0.97 ± 0.04 | 0.54 ± 0.06 |
| PC | 6 | 2.3 ± 0.1 | 7.4 ± 0.1 | 1.02 ± 0.24 | 0.32 ± 0.14* |
| PC + AMP (L) | 7 | 2.1 ± 0.0 | 6.9 ± 0.3 | 1.13 ± 0.08 | 0.07 ± 0.04*# |
| AMP (L) | 6 | 2.1 ± 0.0 | 7.1 ± 0.1 | 1.15 ± 0.11 | 0.37 ± 0.03* |
| AMP (E&L) | 6 | 2.2 ± 0.1 | 7.4 ± 0.1 | 1.17 ± 0.12 | 0.26 ± 0.05* |
| | 6 | 2.3 ± 0.1 | 7.2 ± 0.2 | 1.35 ± 0.15 | 0.13 ± 0.07* |
| Cariporide (L) | 6 | 2.1 ± 0.0 | 6.9 ± 0.2 | 1.13 ± 0.14 | 0.60 ± 0.08 |

Mean ± S.E.M.
*p < 0.05 vs. control;
#p < 0.05 vs. PC and AMP
Abbreviations: see Tables 2 and 3

TABLE 3
Hemodynamic data for 60 min ischemia model

| | | | | |
|---|---|---|---|---|
| Baseline | Ischemia | Rep 30′ | Rep 90′ | Rep 180′ |

HR (beats/min)

| | | | | | |
|---|---|---|---|---|---|
| Control | 275 ± 6 | 2273 ± 9 | 260 ± 9 | 263 ± 9 | 258 ± 8 |
| Cariporide (E) | 286 ± 8 | 267 ± 26 | 274 ± 11 | 272 ± 7 | 263 ± 11 |
| Cariporide (E) + AMP (L) | 277 ± 12 | 269 ± 12 | 238 ± 12 | 239 ± 13 | 246 ± 15 |
| Cariporide (L) + AMP (L) | 272 ± 11 | 260 ± 12 | 235 ± 9 | 240 ± 10 | 243 ± 10 |
| AMP (L) | 289 ± 12 | 274 ± 11 | 235 ± 10 | 253 ± 12 | 268 ± 10 |
| MAP (mmHg) | | | | | |
| Control | 94.3 ± 2.3 | 83.5 ± 3.7 | 78.2 ± 4.1 | 77.7 ± 4.3 | 75.3 ± 4.6 |
| Cariporide (E) | 94.2 ± 1.5 | 86.7 ± 2.1 | 82.8 ± 2.2 | 79.7 ± 2.2 | 79.0 ± 3.3 |
| Cariporide (E) + AMP (L) | 93.8 ± 2.1 | 82.6 ± 2.2 | 68.6 ± 5.1 | 73.0 ± 2.7 | 74.8 ± 3.0 |
| Cariporide (L) + AMP (L) | 93.9 ± 2.3 | 80.0 ± 3.5 | 63.9 ± 4.8 | 67.8 ± 2.5 | 72.8 ± 3.8 |
| AMP (L) | 102.1 ± 2.5 | 93.4 ± 2.9 | 73.3 ± 2.8 | 83.6 ± 4.0 | 85.0 ± 2.9 |

Mean ± S.E.M.
Abbreviations:
Cariporide (E) = a bolus injection of cariporide 5 min prior to ischemia;
Cariporide (E) + AMP (L) = a bolus injection of cariporide 5 min prior to ischemia followed by administration of AMP 579 starting at reperfusion for 70 min;
Cariporide (L) + AMP (L) = a bolus injection of cariporide 5 min followed by administration of AMP 579 starting at reperfusion for 70 min;
AMP (L) = administration of AMP 579 starting at reperfusion and lasting for 70 min;
HR = heart rate;
MAP = mean arterial pressure.

TABLE 4
Infarct size data for 60 min ischemia model

| | | Body weight | Heart weight | Risk zone | Infarct size |
|---|---|---|---|---|---|
| | n | (kg) | (g) | (cm³) | (cm³) |
| Control | 10 | 2.3 ± 0.0 | 7.9 ± 0.2 | 1.14 ± 0.13 | 0.77 ± 0.11 |
| Cariporide (E) | 6 | 2.4 ± 0.0 | 7.6 ± 0.3 | 1.17 ± 0.11 | 0.47 ± 0.08* |
| Cariporide (E) + AMP | 7 | 2.3 ± 0.0 | 7.4 ± 0.1 | 1.03 ± 0.11 | 0.15 ± 0.05*# |
| Cariporide (L) + AMP | 6 | 2.4 ± 0.0 | 7.5 ± 0.2 | 1.09 ± 0.08 | 0.36 ± 0.09* |
| AMP | 8 | 2.3 ± 0.0 | 7.5 ± 0.2 | 1.25 ± 0.12 | 0.59 ± 0.09 |

Mean ± S.E.M.
*p < 0.05 vs. control;
p < 0.05 vs. Cariporide (E) and Cariporide (L) + AMP.
Abbreviation: see Table 1;
n = number of rabbits in each group

An embodiment according to the invention is the use of pharmaceutically effective amounts of a compound having adenosine A1/A2 agonistic activity and a compound having sodium-hydrogen exchanger inhibitory activity in the preparation of a medicament for providing cardioprotection in a patient in need thereof.

A preferred embodiment according to the invention is a pharmaceutical composition comprising a pharmaceutically acceptable carrier and pharmaceutically effective amounts of a compound having adenosine A1/A2 agonistic activity and a sodium-hydrogen exchanger inhibitory compound, wherein the compound having adenosine A1/A2 agonistic activity is AMP 579 or a pharmaceutically acceptable salt thereof.

Another preferred embodiment according to the invention is a pharmaceutical composition comprising a pharmaceutically acceptable carrier and pharmaceutically effective amounts of a compound having adenosine A1/A2 agonistic activity and a sodium-hydrogen exchanger inhibitory compound, wherein the sodium-hydrogen exchanger inhibitory compound is cariporide, eniporide, zoniporide, BMS-284640, BIIB-513, BIIB-722CI, EMD-85131, KB-R9032, MS-31-038, SL-59.1227, SM20550, SMP-300, T-559 and TY-12533.

A more preferred embodiment according to the invention is a pharmaceutical composition comprising a pharmaceutically acceptable carrier and pharmaceutically effective amounts of a compound having adenosine A1/A2 agonistic activity and a sodium-hydrogen exchanger inhibitory compound, wherein the sodium-hydrogen exchanger inhibitory compound is cariporide.

A special embodiment of the invention is a pharmaceutical composition comprising a pharmaceutically acceptable carrier, AMP579 or a pharmaceutically acceptable salt thereof, and cariporide.

Another preferred embodiment according to the invention provides a method of protecting against reperfusion injury in a patient in need thereof comprising administering to said patient pharmaceutically effective amounts of a compound having adenosine A1/A2 agonistic activity and a sodium-hydrogen exchanger inhibitory compound.

Another preferred embodiment according to the invention provides a method of protecting against ischemic injury in a patient in need thereof comprising administering to said patient pharmaceutically effective amounts of a compound having adenosine A1/A2 agonistic activity and a sodium-hydrogen exchanger inhibitory compound.

Another preferred embodiment according to the invention provides a method of providing cardioprotection prior to, during, or following cardiac surgery in a patient in need thereof comprising administering to said patient pharmaceutically effective amounts of a compound having adenosine A1/A2 agonistic activity and a sodium-hydrogen exchanger inhibitory compound.

Another preferred embodiment according to the invention provides a method of providing cardioprotection in a patient in need thereof prior to, during, or following ischemic attack comprising administering to said patient pharmaceutically effective amounts of a compound having adenosine A1/A2 agonistic activity and a sodium-hydrogen exchanger inhibitory compound.

In the cardioprotection method according to the invention the adenosine A1/A2 agonistic compound and sodium-hydrogen exchanger inhibitory compound may be administered in different ways, such as in combination therapies optionally employing medical procedures. For example, the adenosine A1/A2 agonistic compound and sodium-hydrogen inhibitory compound may be administered to a patient concomitantly or at different times provided that they are administered such that at some period of time there are pharmaceutically effective amounts of both compounds present in the patient such that a therapeutic effect according to the invention results.

It is a further object of the invention to provide a kit for providing cardioprotection in a patient, said kit comprising a plurality of separate containers, wherein at least one of said containers contains a compound having adenosine A1/A2 agonistic activity and at least another of said containers contains a sodium-hydrogen exchanger inhibitory compound, and said containers optionally contain a pharmaceutical carrier, which kit may be effectively utilized for carrying out combination therapies according to the invention. A further embodiment for a kit would be wherein of said containers at least one of said containers should contain the compound having adenosine A1/A2 agonistic activity without the presence of the sodium-hydrogen exchanger inhibitory compound, and at least another of said containers should contain the sodium-hydrogen exchanger inhibitory compound without the presence of the compound having adenosine A1/A2 agonistic activity.

In practice, the adenosine A1/A2 agonistic compound and sodium-hydrogen exchanger inhibitory compound may be administered parenterally, topically, rectally, transdermally, intrapulmonary or orally, but they are preferably administered parenterally and/or orally.

Suitable compositions containing the compounds used according to the invention may be prepared by conventional means. For example, the compounds used according to the invention may be dissolved or suspended in a suitable carrier.

The compounds used according to the invention should be presented in forms permitting administration by the most suitable route, and the invention also relates to a pharmaceutical composition containing the compounds used according to the invention which are suitable for use in human or veterinary medicine. These compositions may be prepared according to the customary methods, using one or more pharmaceutically acceptable carrier, which comprise adjuvants or excipients. The adjuvants comprise, inter alia, diluents, sterile aqueous media and the various non-toxic organic solvents. The compositions may be presented in the form of tablets, pills, capsules, lozenges, troches, hard candies, granules, powders, aqueous solutions or suspensions, injectable solutions, elixirs or syrups, powders, solution or suspension for intrapulmonary administration and can contain one or more agents chosen from the group comprising sweeteners, flavorings, colorings, or stabilizers in order to obtain pharmaceutically acceptable preparations.

The choice of vehicle and the content of compounds used according to the invention in the vehicle are generally determined in accordance with the solubility and chemical properties of the compounds, the particular mode of administration and the provisions to be observed in pharmaceutical practice. For example, excipients such as sterile water, Ringer's solution, lactose, sodium citrate, isotonic saline solutions (monosodium or disodium phosphate, sodium, potassium, calcium or magnesium chloride, or mixtures of such salts), calcium carbonate and disintegrating agents such as starch, alginic acids and certain complex silicates combined with lubricants such as magnesium stearate, sodium lauryl sulfate and talc may be used for preparing tablets. To prepare a capsule, it is advantageous to use lactose and high molecular weight polyethylene glycols. When aqueous suspensions are used they can contain emulsifying agents or agents which facilitate suspension. Diluents such as sucrose, ethanol, polyethylene glycol, propylene glycol, glycerol and chloroform or mixtures thereof may also be used.

For parenteral administration, emulsions, suspensions or solutions of the compounds used according to the invention in vegetable oil, for example sesame oil, groundnut oil or olive oil, or aqueous-organic solutions such as water and propylene glycol, injectable organic esters such as ethyl oleate, as well as sterile aqueous solutions of the pharmaceutically acceptable salts, are useful. The solutions of the salts of the compounds used according to the invention are especially useful for administration by intramuscular, intravenous, intraarterial or subcutaneous injection or infusion techniques. The aqueous solutions, also comprising solutions of the salts in pure distilled water, may be used for intravenous administration with the proviso that their pH is suitably adjusted, that they are judiciously buffered and rendered isotonic with a sufficient quantity of glucose or sodium chloride and that they are sterilized by heating, irradiation or microfiltration.

The compound having adenosine A1/A2 agonistic activity and the sodium-hydrogen exchanger inhibitory compound according to the invention may also be formulated in a manner which resists rapid clearance from the vascular (arterial or venous) wall by convection and/or diffusion, thereby increasing the residence time of the composition at the desired site of action. Depot useful according to the invention may be in a copolymer matrix, such as ethylene-vinyl acetate, or a polyvinyl alcohol gel surrounded by a Silastic shell. Alternatively, the compound having adenosine A1/A2 agonistic activity and the sodium-hydrogen exchanger inhibitory compound may be delivered locally from a silicone polymer implanted in the adventitia.

An alternative approach for minimizing washout of the compound having adenosine A1/A2 agonistic activity and the sodium-hydrogen exchanger inhibitory compound during percutaneous, transvascular delivery comprises the use of nondiffusible, drug-eluting microparticles. The microparticles may be comprised of a variety of synthetic polymers, such as polylactide for example, or natural substances, including proteins or polysaccharides. Such microparticles enable strategic manipulation of variables including total dose of a drug and kinetics of its release. Microparticles can be injected efficiently into the arterial or venous wall through a porous balloon catheter or a balloon over stent, and are retained in the vascular wall and the periadventitial tissue for at least about two weeks. Formulations and methodologies for local, intravascular site-specific delivery of therapeutic agents are discussed, for example, in Reissen et al. (J. Am. Coll. Cardiol. 1994; 23: 1234-1244), the entire contents of which are hereby incorporated by reference.

The medium for the compound having adenosine A1/A2 agonistic activity and the sodium-hydrogen exchanger inhibitory compound can also be a hydrogel which is prepared from any biocompatible or non-cytotoxic (homo or hetero) polymer, such as a hydrophilic polyacrylic acid polymer that can act as a drug absorbing sponge. Such polymers have been described, for example, in application WO93/08845, the entire contents of which are hereby incorporated by reference. Certain of them, such as, in particular, those obtained from ethylene and/or propylene oxide are commercially available.

In addition, the compound having adenosine A1/A2 agonistic activity and the sodium-hydrogen exchanger inhibitory compound may be administered directly to the blood vessel wall by means of an angioplasty balloon which is coated with a hydrophilic film (for example a hydrogel), or by means of any other catheter containing an infusion chamber for the compounds, which can thus be applied in a precise manner to the site to be treated.

The percentage of the adenosine A1/A2 agonistic compound and sodium-hydrogen exchanger inhibitory compound used according to the invention may be varied. The compounds should constitute a proportion such that a suitable dosage shall be obtained. Obviously, several unit dosage forms may be administered. The dose employed will be determined by the physician, and depends upon the desired therapeutic effect, the route of administration and the duration of the treatment, and the condition of the patient. In each particular case, the doses will be determined in accordance with the factors distinctive to the subject to be treated, such as age, weight, general state of health and other characteristics which can influence the efficacy of the medicinal product.

In the adult, the dosages of the adenosine A1/A2 agonistic compound are generally from about 0.00001 to about 0.5, preferably about 0.0001 to about 0.05, mg/kg body weight per day by inhalation, from about 0.0001 to about 1, preferably 0.001 to 0.5, mg/kg body weight per day by oral administration, and from about 0.00001 to about 0.1, preferably 0.0001 to 0.01, mg/kg body weight per day by intravenous administration. The dosages of the sodium-hydrogen exchanger inhibitory compound are generally from about 0.0001 to about 5, preferably about 0.001 to about 0.5, mg/kg body weight per day by inhalation, from about 0.001 to about 10, preferably 0.01 to 5, mg/kg body weight per day by oral administration, and from about 0.0001 to about 1, preferably 0.001 to 0.1, mg/kg body weight per day by intravenous administration.

The compound having adenosine A1/A2 agonistic activity and the sodium-hydrogen exchanger inhibitory compound may be administered in dosages which are pharmaceutically effective for each compound, or in dosages which are sub-clinical, i.e., less than pharmaceutically effective for each, or a combination thereof, provided that the combined dosages are pharmaceutically effective.

The compound having adenosine A1/A2 agonistic activity and the sodium-hydrogen exchanger inhibitory compound used according to the invention may be administered as frequently as necessary in order to obtain the desired therapeutic effect. The dosage regimen in carrying out the method of this invention is that which insures maximum therapeutic response until improvement is obtained and thereafter the minimum effective level which gives relief. Some patients may respond rapidly to a higher or lower dose and may find much lower maintenance doses adequate. Both short- and long-term treatments regimens are contemplated for the invention. Treatments at the rate of about 1 to about 4 doses per day are also contemplated, in accordance with the physiological requirements of each particular patient, bearing in mind, of course, that in selecting the appropriate dosages in any specific case, consideration must be given to the patient's weight, general health, age, and other factors which may influence response to the drug. Continuous parenteral infusion, in order to maintain therapeutically effective blood levels of the compound having adenosine A1/A2 agonistic activity and the sodium-hydrogen exchanger inhibitory compound is also contemplated.

The compounds of the present invention may be used during the treatment of restenosis during angioplasty using any device such as balloon, ablation or laser techniques, in order to reduce or protect against injury during reperfusion.

The compounds of the present invention may be used during the treatment of restenosis, in order to reduce or protect against injury during reperfusion, in combination with any anticoagulant, antiplatelet, antithrombotic or profibrinolytic agent. Often patients are concurrently treated prior, during and after interventional procedures with agents of these classes either in order to safely perform the interventional procedure or to prevent deleterious effects of thrombus formation. Some examples of classes of agents known to be anticoagulant, antiplatelet, antithrombotic or profibrinolytic agents include any formulation of thrombin inhibitors or Factor VIIa inhibitors. Some examples of classes of agents known to be anticoagulant, antiplatelet, antithrombotic or profibrinolytic agents include any formulation of aspirin, direct thrombin inhibitors, direct Factor Xa inhibitors, or Factor VIIa inhibitors.

The present invention may be embodied in other specific forms without departing from the spirit or essential attributes thereof.

We claim:

1. A pharmaceutical composition comprising a pharmaceutically acceptable carrier, pharmaceutically effective amounts of a compound having adenosine A1/A2 agonistic activity, and a sodium-hydrogen exchanger inhibitory compound.

2. The pharmaceutical composition according to claim 1 wherein the compound having adenosine A1/A2 agonistic activity is a compound of the formula

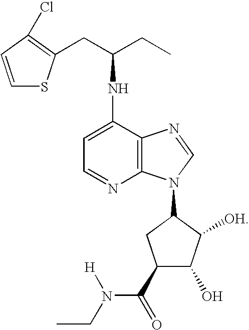

3. The pharmaceutical composition according to claim 1 wherein the sodium-hydrogen exchanger inhibitory compound is selected from the group consisting of cariporide, eniporide, zoniporide, BMS-284640, BIIB-513, BIIB-722CI, EMD-85131, KB-R9032, MS-31-038, SL-59.1227, SM20550, SMP-300, T-559 and TY-12533.

4. The pharmaceutical composition according to claim 1 wherein the sodium-hydrogen exchanger inhibitory compound is cariporide.

5. The pharmaceutical composition according to claim 1 wherein the compound having adenosine A1/A2 agonistic activity is AMP579 and the sodium-hydrogen exchanger inhibitory compound is cariporide.

6. A method of providing cardioprotection in a patient in need thereof comprising administering to said patient the pharmaceutical composition according to claim 1.

7. A method of protecting against reperfusion injury in a patient in need thereof comprising administering to said patient the pharmaceutical composition according to claim 1.

8. A method of protecting against ischemic injury in a patient in need thereof comprising administering to said patient the pharmaceutical composition according to claim 1.

9. A method of providing cardioprotection prior to, during, or following cardiac surgery in a patient in need thereof comprising administering to said patient the pharmaceutical composition according to claim 1.

10. A method of providing cardioprotection in a patient in need thereof prior to, during, or following ischemic attack comprising administering to said patient the pharmaceutical composition according to claim 1.

11. A kit for providing cardioprotection in a patient in need thereof, said kit comprising a plurality of separate containers, wherein at least one of said containers contains a compound having adenosine A1/A2 agonistic activity and at least another of said containers contains a sodium-hydrogen exchanger inhibitory compound, and said containers optionally contain a pharmaceutical carrier.

12. A kit according to claim 11 wherein of said containers at least one of said containers should contain the compound having adenosine A1/A2 agonistic activity without the presence of sodium-hydrogen exchanger inhibitory compound, and at least another of said containers should contain the sodium-hydrogen exchanger inhibitory compound without the presence of the compound having adenosine A1/A2 agonistic activity.

13. A pharmaceutical composition comprising a pharmaceutically acceptable carrier and a pharmaceutically effective amount, or less than pharmaceutically effective amount of a compound having adenosine A1/A2 agonistic activity and a pharmaceutically effective amount, or less than a pharmaceutically effective amount of a sodium-hydrogen exchanger inhibitory compound, provided that the composition is pharmaceutically effective.
